(19) United States
(12) Patent Application Publication
(10) Pub. No.: US 2026/0193603 A1
(43) Pub. Date: Jul. 9, 2026

(54) METHOD FOR SELECTING EMBRYO

(71) Applicants: NATIONAL UNIVERSITY CORPORATION TOKYO UNIVERSITY OF AGRICULTURE AND TECHNOLOGY, Tokyo (JP); NATIONAL LIVESTOCK BREEDING CENTER INCORPORATED ADMINISTRATIVE AGENCY, Nishishirakawa-gun, Fukushima (JP); KAWATA ANIMAL CLINIC, Fukaya-shi, Saitama (JP)

(72) Inventors: Satoshi SUGIMURA, Tokyo (JP); Satoko MATOBA, Nishishirakawa-gun, Fukushima (JP); Ryusaku KAWATA, Fukaya-shi, Saitama (JP)

(73) Assignees: NATIONAL UNIVERSITY CORPORATION TOKYO UNIVERSITY OF AGRICULTURE AND TECHNOLOGY, Tokyo (JP); NATIONAL LIVESTOCK BREEDING CENTER INCORPORATED ADMINISTRATIVE AGENCY, Nishishirakawa-gun, Fukushima (JP); KAWATA ANIMAL CLINIC, Fukaya-shi, Saitama (JP)

(21) Appl. No.: 19/134,317

(22) PCT Filed: Nov. 30, 2023
(86) PCT No.: PCT/JP2023/042931

§ 371 Date: May 30, 2025

(30) Foreign Application Priority Data
Dec. 1, 2022 (JP) 2022-192945

(51) Int. Cl. A01K 67/02; C12N 5/073

(52) U.S. Cl. CPC A01K 67/02; C12N 5/0604; A01K 2227/101

(57) ABSTRACT

This invention provides a method for obtaining a mammalian embryo having a high conception rate in a simple manner. When selecting a mammalian embryo prepared by in vitro culture from a fertilized egg, an embryo with an appearance that satisfies at least one of Index 1 to Index 3 below is selected: Index 1: a secretion product is not observed in the vicinity of an embryo at the blastocyst stage; Index 2: partial compaction is not observed at the morula stage; and Index 3; none of direct cleavage (DC), abnormal cytokinesis (AC), fragmentation (F), and unequal sizes among sister blastomeres (UB) is observed at a stage after first cleavage and before second cleavage.

### TECHNICAL FIELD

The present invention relates to a method for selecting embryos having high conception rates from among mammalian embryos obtained by in vitro culture, a method for producing embryos having high conception rates from mammalian fertilized eggs by in vitro culture, and a method for producing mammals using embryos obtained by these methods.

### BACKGROUND ART

Technology has been established for many mammals such as cattle, whereby fertilized eggs are obtained by in vitro fertilization, and embryos are developed from fertilized eggs by in vitro culture. The obtained embryos are implanted into the uteri of recipient females for conception, and progeny are thus obtained. However, embryos obtained by in vitro fertilization are problematic due to low conception rates. For example, the conception rates of cattle range from about 40% to 50%, and human pregnancy success rates range from 25% to 35%. Possible causes thereof may be, for example, poor development of embryos since in vitro culture environments differ from in vivo environments. Accordingly, development of techniques for selecting embryos having high conception rates based on morphology or biochemical indices has been attempted.

Non-patent literature 1 reports that, in human fertilized eggs, conception rates differ depending on the number of cells and fragmentation at the time of third cleavage. Non-patent literature 2 reports that, in human fertilized eggs, the conception rates are improved when there are few instances of fragmentation at the time of third cleavage. Non-patent literatures 3 and 4 report that, in bovine fertilized eggs, when the number of cells at the time of third cleavage is not 5 to 8 cells, chromosome aberration takes place particularly easily. Non-patent literature 5 reports that, in bovine fertilized eggs, conception rates fluctuate depending on respiratory volumes (the amount of oxygen consumed), and a respiratory volume of 0.78-1.10 nl/h leads to the highest conception rate. Non-patent literature 6 describes the examination of the relationship between combinations of a plurality of indices (e.g., the number of cells at the time of initial cleavage, the time required for initial cleavage to take place, the uniformity of 2-cell embryos at the time of initial cleavage, the number of cells at the time of second cleavage, amino acid level, and the like in porcine fertilized eggs) and blastocyst (%).

Patent literature 1 discloses an invention relating to a method for evaluating embryo quality. The literature describes that unsynchronized cell division at the time of cleavage and fragmentation phenomenon can be used as indices for quality evaluation. The literature further describes that these indices may be combined with other indices such as respiration rate. Patent literature 2 discloses an apparatus and a method for measuring the amount of oxygen consumed per single embryo in order to evaluate embryo quality.

Patent literature 3 and Non-patent literature 7 disclose culture vessels that are suitable for observation by time-lapse imaging at the time of fertilized egg culture. Patent literature 4 and Non-patent literatures 8 and 9 disclose methods for evaluating mammalian embryos based on morphological dynamics using a time-lapse image shooting apparatus (the timing of first cleavage, the number of blastomeres upon completion of first cleavage, the occurrence of fragmentation at the time of first cleavage, and the number of blastomeres at the temporary resting stage of embryos) and the amount of oxygen consumed by blastocysts.

### CITATION LIST

### Patent Literature

Patent literature 1: JP 2009-539387 A
Patent literature 2: JP 2002-122568 A
Patent literature 3: JP 2010-000748 A
Patent literature 4: JP 2016-168059 A

### Non-Patent Literature

Non-patent literature 1: Human Reproduction, Vol. 16, No. 9, pp. 1970-1975, 2001
Non-patent literature 2: Human Reproduction, Vol. 17, No. 9, pp. 2402-2409, 2002
Non-patent literature 3: BIOLOGY OF REPRODUCTION, Vol. 63, 1143-1148, 2000
Non-patent literature 4: J. Reprod. Dev., Vol. 54, No. 6, 465-472, 2008
Non-patent literature 5: Human Reproduction, Vol. 22, No. 2, pp. 558-566, 2007
Non-patent literature 6: BIOLOGY OF REPRODUCTION, Vol. 77, 765-779, 2007
Non-patent literature 7: Biol. Reprod., Vol. 83, No. 6:970-8, 2010
Non-patent literature 8: PLOS One, 2012; 7 (5): e36627
Non-patent literature 9: J. Reprod. Dev., Vol. 63, No. 4:353-357, 2017

### SUMMARY OF INVENTION

### Technical Problem

An object of the present invention is to provide a method for readily obtaining mammalian embryos having high conception rates.

### Solution to Problem

The present invention provides the following.
(1) A method for selecting a mammalian embryo prepared by in vitro culture from a fertilized egg comprising a step of selecting an embryo, provided that an embryo's appearance satisfies at least one of the Index 1 to Index 3 below:
   Index 1: a secretion product is not observed in the vicinity of an embryo at the blastocyst stage;
   Index 2: partial compaction is not observed at the morula stage; and
   Index 3: none of direct cleavage (DC), abnormal cytokinesis (AC), fragmentation (F), and unequal blastomeres (UB) is observed at a stage after first cleavage and before second cleavage.
(2) The method according to (1), wherein the step of selecting an embryo satisfies Index 1.
(3) The method according to (2), wherein the step of selecting an embryo further satisfies Index 2.
(4) The method according to (2), wherein the step of selecting an embryo further satisfies Index 3.
(5) The method according to any of (1) to (4), wherein the step of selecting an embryo satisfies all of Index 1 to Index 3.
(6) The method according to any of (1) to (5), wherein the mammal is cattle.
(7) A method for producing an embryo from a mammalian fertilized egg comprising:
   a step of in vitro culture of a fertilized egg; and
   at least one of Step a to Step c below:
   Step a: a step of selecting an embryo that does not have a secretion product in the vicinity thereof at the blastocyst stage;
   Step b: a step of selecting an embryo that does not undergo partial compaction at the morula stage; and
   Step c: a step of selecting an embryo that does not undergo any of DC, AC, F, or UB at a stage after first cleavage and before second cleavage.
(8) The method according to (7), which comprises Step a.
(9) The method according to (8), which further comprises Step b.
(10) The method according to (8), which further comprises Step c.
(11) The method according to any of (7) to (10), which comprises all of Step a to Step c.
(12) The method according to any of (7) to (11), wherein the mammal is cattle.
(13) A method for producing an individual mammal comprising a step of implanting an embryo prepared by the method according to any of (7) to (12) into an individual female for conception.
(14) An embryo selection apparatus for selecting a mammalian embryo prepared by in vitro culture from a fertilized egg comprising:
   an image shooting apparatus for obtaining an image of an embryo; and
   an analysis section for analyzing the obtained image,
   wherein the analysis section selects an embryo suitable for implantation based on the obtained image, provided that the embryo satisfies at least one of Index 1 to Index 3 below.
   Index 1: a secretion product is not observed in the vicinity of an embryo at the blastocyst stage;
   Index 2: partial compaction is not observed at the morula stage; and
   Index 3: none of DC, AC, F, and UB is observed at a stage after first cleavage and before second cleavage.

The description incorporates the contents disclosed by JP Patent Application No. 2022-192945, based on which the priority of the present application claims.

### Advantageous Effects of Invention

According to the method of the present invention, mammalian embryos having high conception rates can be readily obtained.

### BRIEF DESCRIPTION OF DRAWINGS

FIG. 1 schematically shows examples of abnormal cleavage after first cleavage of mammalian fertilized eggs. “DC” stands for direct cleavage, “AC” stands for abnormal cytokinesis, “F” stands for fragmentation, and “UB” stands for unequal blastomeres.

FIG. 2 shows the outline of a general change in appearance from the mammalian fertilized egg to the blastocyst stage.

FIG. 3 shows a photograph showing an example of a bovine blastocyst that has generated a secretion product. A part indicated with an arrow is a secretion product.

FIG. 4 shows a photograph showing a bovine embryo at the morula stage that undergoes partial compaction. A region surrounded by a broken line is a site of partial compaction.

FIG. 5 shows a chart showing conception rates of 370 samples of bovine fertilized eggs tested in the examples that are classified into: a group of embryos not satisfying Index 1 (a group of embryos “with” a secretion product); and a group of embryos satisfying Index 1 (a group of embryos “without” a secretion product). In FIG. 5, the symbol “*” indicates that the p-value exceeds 0.05 in a χ² test (P<0.05).

FIG. 6 shows a chart showing conception rates of 370 samples of bovine fertilized eggs tested in the examples that are classified into: a group of embryos not satisfying Index 2 (a group of embryos “with” PC); and a group of embryos satisfying Index 2 (a group of embryos “without” PC). In FIG. 6, the symbol “*” indicates that the p-value exceeds 0.05 in a χ² test (P<0.05).

FIG. 7 shows a chart showing conception rates of 370 samples of bovine fertilized eggs tested in the examples that are classified into: a group of embryos that undergo DC, AC, F, and UB after first cleavage (a group of embryos “+”); and a group of embryos that do not undergo DC, AC, F, and UB (a group of embryos “−”). In FIG. 7, the symbol “*” indicates that the p-value exceeds 0.05 in a χ² test (P<0.05).

FIG. 8 schematically shows the data indicating conception rates of 370 samples of bovine fertilized eggs tested in the examples that are evaluated in the order from Index 3, Index 2, to Index 1.

### DESCRIPTION OF EMBODIMENTS

### [1] Outline and Definition

The present inventors produced embryos for implantation by in vitro culture of mammalian fertilized eggs and discovered that embryos exhibiting a characteristic appearance at a particular developmental stage would have low conception rates. This has led to the completion of the present invention aimed at selection of embryos having higher conception rates.

The term “mammals” used herein refers to warm blooded vertebrates. Examples thereof include primates such as humans and monkeys, rodents such as mice, rats, and rabbits, pet animals such as dogs and cats, and livestock animals such as cattle, horses, pigs, and sheep. The method of the present invention is typically used for non-human mammals (mammals other than humans). In the present invention, the term “humans” refers to Homo sapiens. The term “monkeys” refers to non-human animals that are classified as members of the Order Primates. The term “mice” refers to Mus musculus. The term “rats” refers to Rattus norvegicus. The term “rabbits” refers to animals classified as members of the family Leporidae. The term “dogs” refers to animals classified as Canis lupus, and typically refers to Canis lupus familiaris. The term “cats” refers to animals classified as Felis silvestris, and typically refers to Felis silvestris catus. The term “cattle” refers to animals classified as members of the genus Bos, and typically refers to Bos Taurus and Bos indicus. The term “horses” refers to Equus caballus. The term “pigs” refers to animals classified as Sus scrofa, and typically refers to Sus scrofa domesticus. The term “sheep” refers to Ovis aries.

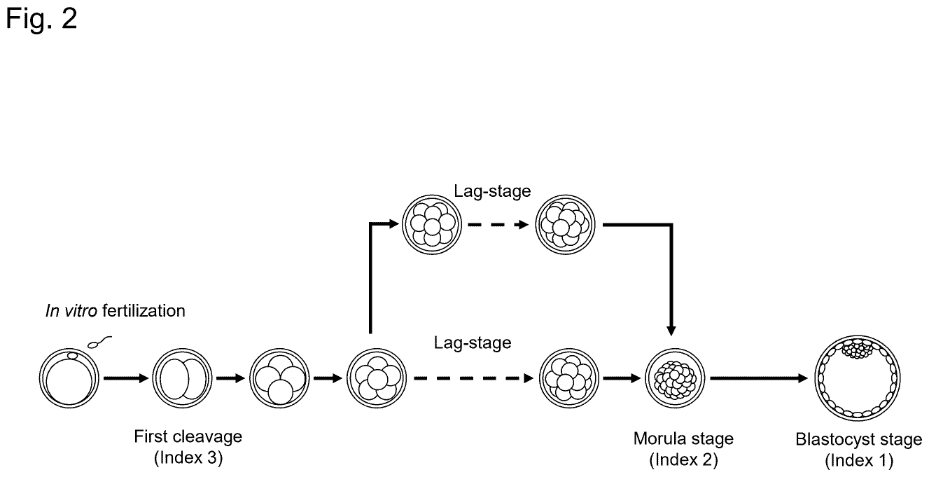

FIG. 2 shows the outline of a general change in appearance from the mammalian fertilized egg to the blastocyst stage. A mammalian fertilized egg divides into a 2-cell stage, a 4-cell stage, and then an 8-cell stage via cleavage after fertilization, so that the number of cells increases. In the case of a normal fertilized egg, in general, a fertilized egg is substantially equally segmented to this stage. Upon completion of the 8-cell stage, configurations of blastomeres change, blastomeres are brought into contact with one another from the 16-cell stage, the 32-cell stage, to the morula stage, and blastomeres form a single mass that makes difficult to distinguish the boundary between blastomeres. This phenomenon is referred to as “compaction.” After the 32-cell stage, outer cells absorb moisture to form a single cell layer (trophectoderm), and inner cell masses are attached to a part inside the cell layer. A stage in such a state is referred to as the “blastocyst stage.”

In general, fertilized eggs obtained by in vitro fertilization are grown to the blastocyst stage by in vitro culture, implanted into individual females, and then generated as new individuals through conception, growing, and birthing. Examples of embryos to be subjected to selection or production herein include a blastocyst, an expanded blastocyst, and a hatched blastocyst. Specific examples include an early blastocyst, a blastocyst, an expanded blastocyst, and a hatched blastocyst. An early blastocyst, a blastocyst, an expanded blastocyst, and a hatched blastocyst are provided with the trophectoderm potentially capable of forming the placenta and the inner cell mass potentially capable of forming embryos.

The terms “early blastocyst,” “blastocyst,” “expanded blastocyst,” and “hatched blastocyst” are defined in Robertson I, Nelson RE, 1998, Certification and identification of the embryo. In: DA Stringfellow and SM Seidel, Editors, Manual of the international embryo transfer society, IETS, Savoy, Illinois, 103-116 and are thus apparent to persons skilled in the art. The terms are as specifically explained below for reference.

The term “early blastocyst” refers to an embryo at a stage when a blastocoele can be observed under a microscope. An early blastocyst exhibits a ring-like form.

When an early blastocyst grows, the separation of cytotrophoblasts proceeds, inner cell masses are darkened, and the former can be clearly distinguished from the latter. The “blastocyst” is thus developed. A blastocoele broadly expands within the perivitelline space and then almost fills the perivitelline space.

The term “expanded blastocyst” refers to an embryo after a blastocyst grows, a blastocoele expands significantly, and the whole size then increases (about 1.2 to 1.5 times the size of an embryo at a stage up to the blastocyst stage) as well as the thickness of the zona pellucida thins to about one-third the original thickness.

The term “hatched blastocyst” refers to an embryo with open zona pellucida that has broken out of its zona pellucida.

The term “blastocyst stage” used herein may refer to any of the early blastocyst, blastocyst, expanded blastocyst, or hatched blastocyst stage. In general, the term refers to the early blastocyst, blastocyst, or expanded blastocyst stage at which an embryo is present inside the zona pellucida.

The term “lag-phase” refers to a stage at which cell division is temporarily arrested. In the case of a fertilized egg, the stage after second cleavage and before third cleavage, the stage after third cleavage and before fourth cleavage, or the stage after fourth cleavage and before fifth cleavage is equivalent to the lag-phase.

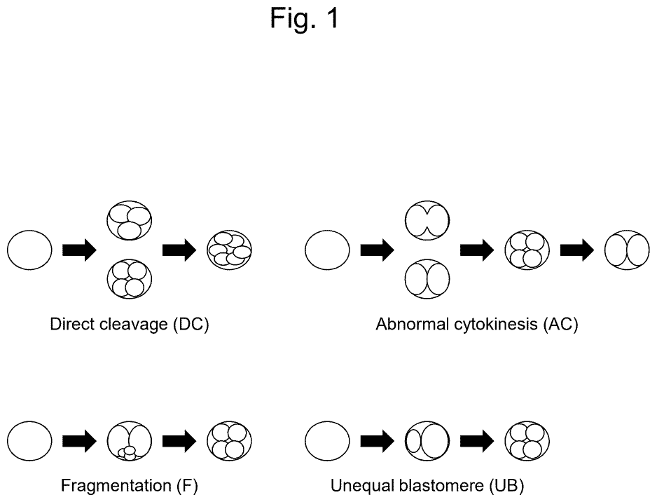

The phenomena referred to as direct cleavage (DC), abnormal cytokinesis (AC), fragmentation (F), and unequal blastomeres (UB) herein are generally observed at a stage after first cleavage and before second cleavage. FIG. 1 shows examples of fertilized eggs in the states of DC, AC, F, and UB. DC is an abbreviation of direct cleavage, which is a phenomenon where one cell divides into three or more cells in a single instance of cell division. AC is an abbreviation of abnormal cytokinesis, which is a phenomenon where divided cells are fused again to form a single cell. F is an abbreviation of fragmentation, which is a phenomenon where fragmented cells (cytoplasmic globules) are observed in addition to the cleaved cells. Concerning “F,” whether or not a nucleus is contained in fragmented cells (cytoplasmic globules) is irrelevant herein. “F” encompasses a phenomenon where an fertilized egg has a plurality of cell fragments and a phenomenon where a cell protrusion with a size different from that of a blastomere is generated. UB is an abbreviation of unequal blastomere, which is a phenomenon where diameters of two cells divided from a single cell differ from each other by 25% or more.

### [2] a Method for Selecting a Mammalian Embryo Prepared by In Vitro Culture from a Fertilized Egg

The first embodiment of the present invention relates to a method for selecting a mammalian embryo prepared by in vitro culture from a fertilized egg. The method of the present embodiment comprises a step of selecting an embryo, provided that an embryo's appearance satisfies at least one of Index 1 to Index 3 below:
Index 1: a secretion product is not observed in the vicinity of an embryo at the blastocyst stage;
Index 2: partial compaction is not observed at the morula stage; and
Index 3: none of direct cleavage (DC), abnormal cytokinesis (AC), fragmentation (F), and unequal sizes among sister blastomeres (UB) is observed at a stage after first cleavage and before second cleavage.

In the method of the present embodiment, it is preferable that the step of selecting an embryo satisfy Index 1. In the method of the present embodiment, it is more preferable that the step of selecting an embryo satisfy Index 1 in combination with Index 2. In the method of the present embodiment, alternatively, it is more preferable that the step of selecting an embryo satisfy Index 1 in combination with Index 3. In the method of the present embodiment, it is further preferable that the step of selecting an embryo satisfy all of Index 1 to Index 3.

A fertilized egg used in the method of the present embodiment is not particularly limited, provided that it is a mammalian fertilized egg. Use of a bovine fertilized egg is particularly preferable. A method for obtaining a fertilized egg is not particularly limited. It is possible to prepare a fertilized egg by in vitro fertilization of an egg cell with sperm.

As for various conditions such as a medium, temperature, and the composition of atmospheric gas to be used for culturing a fertilized egg (an embryo), in the method of the present embodiment, conditions that are generally used can be employed depending on the mammalian species.

As typical culture conditions for cattle embryos, preferably, temperatures ranging from 38.0° C. to 39.5° C. and more preferably ranging from 38.5° C. to 39° C., a medium such as SOF (synthetic oviduct fluid), modified SOF, IVD-101, TCM199, CR1aa, or BO-IVF and gas, such as gas with saturated humidity containing 4.5% to 5.5% CO₂ and the remainder of air (e.g., saturated humidity and 5% CO₂/95% air) or gas with saturated humidity containing 4.5% to 5.5% CO₂, 4.5% to 5.5% CO₂ and the remainder of N₂ (e.g., saturated humidity·5% CO₂/5% O₂/90% N²), can be employed, for example.

All the indices used in the method of the present embodiment are based on an embryo's appearance and that in the vicinity of the embryo. In the method of the present embodiment, specifically, it is necessary to observe an embryo's appearance at a particular stage. Observation of an embryo's appearance can be performed by a noninvasive means such as microscopy. In general, morphology is observed with 40 times to 200 times magnification.

In the method of the present embodiment, it is necessary to observe the appearance of a fertilized egg (an embryo) at an adequate stage during culture thereof. Observation of the appearance in terms of the indices used in the method of the present embodiment is performed at the stages described below:
Index 1: at the blastocyst stage;
Index 2: at the morula stage; and
Index 3: at a stage after first cleavage and before second cleavage.

When all of such indices are used, as shown in FIG. 2, appearance observation is performed in the order from Index 3, Index 2, to Index 1 in a period from culture of fertilized eggs to preparation of embryos for implantation.

The specific timing at each stage varies depending on animal species. A rough time zone is known for the animal species of the same species. In the case of cattle, first cleavage is generally completed 20 to 40 hours after fertilization, and it is typically completed approximately 23 to 33 hours after fertilization. The morula stage is reached 80 to 140 hours after fertilization, and it is typically reached approximately 96 to 122 hours after fertilization. The blastocyst stage is reached 120 to 216 hours after fertilization, and it is typically reached approximately 144 to 192 hours after fertilization. Accordingly, an embryo's appearance is observed at an adequate timing after fertilization depending on the index to be used, so that the appearance can be evaluated in terms of the index of interest.

Concerning the timing of each step of embryonic development, as described above, it is possible to set a rough time zone for the animal species of the same species. It is possible to remove an embryo from a culture tank and obtain an image at each timing for observation. In such a case, however, there are individual differences and variability in the timing at each stage to some extent among animals of even the same species. Therefore, it may not be possible to obtain an image at an adequate timing. When removing an embryo from a culture tank, conditions, such as temperature and humidity, in the vicinity of an embryo may temporarily change. In addition to individual differences, culture and appearance observation can be performed using a culture tank equipped with a time-lapse image shooting apparatus, so as to observe the appearance at an adequate timing under adequate conditions. A time-lapse image shooting apparatus is fixed inside a culture tank, and it obtains images of an embryo at constant time intervals (e.g., approximately 5 to 20 minutes). With the use of a culture tank equipped with a time-lapse image shooting apparatus, an image of an embryo's appearance can be obtained at an adequate timing without removing the embryo from the culture tank.

Evaluation in terms of indices by observation of an embryo's appearance at each stage can be visually performed based on microscopic images. Alternatively, evaluation in terms of indices by observation of an embryo's appearance can be automatically performed based on microscopic images using analytical means such as computers. Analytical means may involve the use of artificial intelligence (AI). When AI is used, a machine-learned model using a known neural network may be constructed with the use of the image databases of embryos satisfying the indices and of embryos not satisfying the indices as teaching data. Analytical means can use such model to evaluate as to whether or not an embryo of interest satisfies the indices based on the images and output the results.

In the method of the present embodiment, known indices for embryo selection may be used in addition to any one of Index 1 to Index 3 or any thereof in combination. An example of a known index is an embryo quality code recommended by the International Embryo Technology Society (IETS). According to the IETS standard, embryo quality is classified into Code 1 to Code 4. It is impossible to use a code 4 embryo for implantation. Accordingly, in practice, embryo quality is graded from Code 1 to code 3:
Code 1: Excellent or good
Code 2: Fair
Code 3: Poor
Code 4: Dead or degenerating.

Alternatively, as known indices, for example, indices described in Patent literature 4 and Non-patent literatures 8 and 9 can also be used.

### [2-1] Index 1

An aspect of the method according to the present embodiment comprises a step of selecting an embryo, provided that an embryo's appearance satisfies Index 1 below:
Index 1: a secretion product is not observed in the vicinity of an embryo at the blastocyst stage.

The present inventors discovered that conception rates achieved by implanting embryos that satisfy Index 1 into individual females would be significantly higher than those achieved by implanting embryos that do not satisfy Index 1. Accordingly, this aspect enables production of embryos having high conception rates.

Index 1 can be evaluated by observation of an embryo's appearance at the blastocyst stage. The timing of appearance observation in terms of Index 1 varies depending on animal species. In the case of cattle, observation may be performed 120 to 216 hours after fertilization, and it may be typically performed approximately 144 to 192 hours after fertilization.

The term “secretion product” as specified in Index 1 is observed in some embryos. The term refers to a particulate substance that is generated in the vicinity of an embryo, in particular, a particulate substance that is generated in a medium in the outer vicinity of the zona pellucida. The term “particulate” used herein refers to an approximately spherical or approximately oval spherical form. The term “vicinity” used herein refers to a position away from the surface of the zona pellucida by a distance of less than an embryo diameter, in particular, a distance of less than 50% of an embryo diameter, or a distance of less than 30% of an embryo diameter.

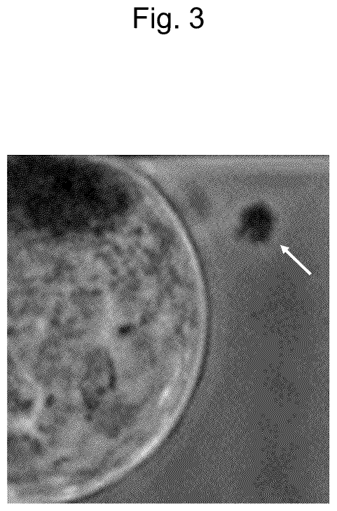

More specifically, the “secretion product” is a particulate part observed in a medium in the vicinity of an embryo when the embryo is observed under an optical microscope that exhibits a color or refractive index different from that in a surrounding medium. FIG. 3 shows a photograph showing an example of an embryo that has generated a secretion product. In FIG. 3, a part indicated with an arrow is a secretion product. A size of a secretion product is not particularly limited, provided that such secretion product can be visually recognized in an optical microscopic image. It is possible to observe a secretion product with a size of 5 to 60 μm, and, in particular, a secretion product with a size of approximately 10 to 45 μm. A size of a blastocyst is generally approximately 140 to 260 μm. Thus, a secretion product is generally observed as a particle with a size that is approximately 1/50 to ⅓ the size of a blastocyst. Index 1 is used to evaluate the presence or absence of a secretion product that is visually recognized in a relatively easy manner. Compared with known indices used for embryo selection, Index 1 enables more objective evaluation.

### [2-2] Index 2

An aspect of the method according to the present embodiment comprises a step of selecting an embryo, provided that an embryo's appearance satisfies Index 2 below:
Index 2: partial compaction is not observed at the morula stage.

The present inventors discovered that conception rates achieved by implanting embryos that satisfy Index 2 into individual females would be significantly higher than those achieved by implanting embryos that do not satisfy Index 2. Accordingly, this aspect enables production of embryos having high conception rates.

It is possible to evaluate in terms of Index 2 by observation of an embryo's appearance at the morula stage. The timing of appearance observation in terms of Index 2 varies depending on animal species. In the case of cattle, observation may be performed 80 to 140 hours after fertilization, and it may be typically performed approximately 96 to 120 hours after fertilization.

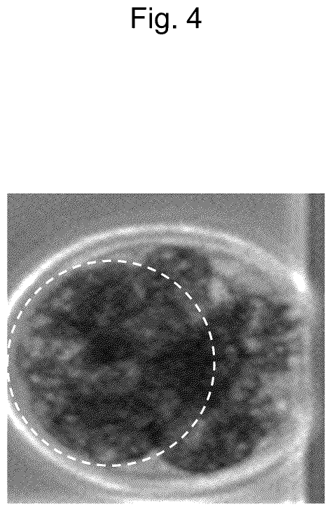

The term “partial compaction” as specified in Index 2 is a phenomenon that is observed in some embryos. In the case of normal embryos, the entire cells form a single mass at the morula stage; i.e., “compaction” occurs. In the case of partial compaction, however, compaction selectively occurs in some part, and other parts are maintained in a manner that cell boundaries can be visually recognized. FIG. 4 shows a photograph showing an embryo at the morula stage that undergoes partial compaction. In FIG. 4, a region surrounded by a broken line is a site of compaction when partial compaction has occurred. In the case of normal embryos, the entire embryos exhibit the appearance indicated by a region surrounded by a broken line.

Index 2 is used to evaluate the occurrence of partial compaction that is visually recognized in a relatively easy manner. Compared with known indices used for embryo selection, Index 2 enables more objective evaluation.

### [2-3] Index 3

An aspect of the method according to the present embodiment comprises a step of selecting an embryo, provided that an embryo's appearance satisfies Index 3 below:

Index 3: none of DC, AC, F, and UB is observed at a stage after first cleavage and before second cleavage.

The present inventors discovered that conception rates achieved by implanting embryos that satisfy Index 3 into individual females would be significantly higher than those achieved by implanting embryos that do not satisfy Index 3. Accordingly, this aspect enables production of embryos having high conception rates.

A normal fertilized egg divides into two cells of substantially the same size at the time of first cleavage. However, some embryos undergo abnormal cleavage, such as DC, AC, F, or UB. FIG. 1 schematically shows the states of DC, AC, F, and UB.

Index 3 can be evaluated by observation of an embryo's appearance at a stage after first cleavage and before second cleavage. The timing of appearance observation in terms of Index 3 varies depending on animal species. In the case of cattle, observation may be performed 20 to 40 hours after fertilization, and it may be typically performed approximately 23 to 33 hours after fertilization.

Index 3 can be evaluated at an early stage after fertilization. Accordingly, Index 3 can be used to refrain from culturing embryos that are not suitable for use for a long period of time.

### [3] a Method for Producing an Embryo from a Mammalian Fertilized Egg

The second embodiment of the present invention relates to a method for producing an embryo from a mammalian fertilized egg. The method of the present embodiment comprises a step of in vitro culture of a fertilized egg and at least one of Step a to Step c:
Step a: a step of selecting an embryo that does not have a secretion product in the vicinity thereof at the blastocyst stage;
Step b: a step of selecting an embryo that does not undergo partial compaction at the morula stage; and
Step c: a step of selecting an embryo that does not undergo any of DC, AC, F, or UB at a stage after first cleavage and before second cleavage.

It is preferable that the method of the present embodiment comprise Step a. It is more preferable that the method of the present embodiment comprise Step a in combination with Step b. Alternatively, it is more preferable that the method of the present embodiment comprise Step a in combination with Step c. It is further preferable that the method of the present embodiment comprise all of Step a to Step c.

A fertilized egg used in the method of the present embodiment is typically a fertilized egg of a non-human mammal (a mammal other than a human). Use of a bovine fertilized egg is particularly preferable. A method for obtaining a fertilized egg is not particularly limited. It is possible to prepare a fertilized egg by in vitro fertilization of an egg cell with sperm.

As for various conditions such as a medium, temperature, and the composition of atmospheric gas to be used for culturing a fertilized egg (an embryo), in the method of the present embodiment, conditions that are generally used can be employed depending on mammal species.

In the method of the present embodiment, Step a to Step c are performed based on an embryo's appearance and that in the vicinity of the embryo. In the method of the present embodiment, specifically, it is necessary to observe an embryo's appearance at a particular stage. Observation of an embryo's appearance can be performed by a noninvasive means such as microscopy. In general, morphology is observed with 40 times to 200 times magnification.

In the method of the present embodiment, it is necessary to observe the appearance of a fertilized egg (an embryo) at an adequate stage during culture thereof. Step a to Step c are performed at the stages described below.
Step a: at the blastocyst stage;
Step b: at the morula stage; and
Step c: at a stage after first cleavage and before second cleavage.

When the method comprises all of the steps, as shown in FIG. 2, appearance observation is performed in the order from Step c, Step b, to Step a in a period from culture of fertilized eggs to preparation of embryos for implantation.

The specific timing, the actual method of culture, the method of observation, and the like at a stage after first cleavage and before second cleavage, the morula stage, and the blastocyst stage are as described in the “[2] A method for selecting a mammalian embryo prepared by in vitro culture from a fertilized egg” section described above.

### [3-1] Step a

An aspect of the method according to the present embodiment comprises Step a described below:
Step a: a step of selecting an embryo that does not have a secretion product in the vicinity thereof at the blastocyst stage.

Step a is performed by observation of an embryo's appearance at the blastocyst stage. The timing of appearance observation varies depending on animal species. In the case of cattle, observation may be performed 120 to 216 hours after fertilization, and it may be typically performed approximately 144 to 192 hours after fertilization.

The definition of the term “secretion product,” the method of observation, and the like are as described in the “[2-1] Index 1” section above.

### [3-2] Step b

An aspect of the method according to the present embodiment comprises Step b described below:
Step b: a step of selecting an embryo that does not undergo partial compaction at the morula stage.

The method of this aspect comprises Step b and thus enables production of embryos having high conception rates.

Step b is performed by observation of an embryo's appearance at the morula stage. The timing of appearance observation varies depending on animal species. In the case of cattle, observation may be performed 80 to 140 hours after fertilization, and it may be typically performed approximately 96 to 122 hours after fertilization.

The definition of the term “partial compaction,” the method of observation, and the like are as described in the “[2-2] Index 2” section above.

### [3-3] Step c

An aspect of mode of the method according to the present embodiment comprises Step c described below:
Step c: a step of selecting an embryo that does not undergo any of DC, AC, F, or UB at a stage after first cleavage and before second cleavage.

The method of the present mode comprises Step c and thus enables production of embryos having high conception rates.

Step c is performed by observation of an embryo's appearance at a stage after first cleavage and before second cleavage. The timing of appearance observation varies depending on animal species. In the case of cattle, observation may be performed 20 to 40 hours after fertilization, and it may be typically performed approximately 23 to 33 hours after fertilization.

A method for observing DC, AC, F, and UB and the like are as described in the “[2-3] Index 3” section above.

### [4] a Method for Producing an Individual Mammal

The third embodiment of the present invention relates to a method for producing an individual mammal. More specifically, the method of the present embodiment comprises a step of implanting an embryo prepared by the method described in the “[3] A method for producing an embryo from a mammalian fertilized egg” section into an individual female for conception. The method of the present embodiment is advantageous in that conception can be achieved with a high probability and an individual mammal can be obtained efficiently.

In the method of the present embodiment, an individual female (recipient) to be subjected to implantation is in a pseudo-pregnancy state (the luteal phase), and an embryo is implanted into the uterine horn, oviduct, or the like of the individual female. After conception, a step of obtaining progeny and a step of obtaining individual animals by growing progeny can be performed by conventional methods.

### [5] an Embryo Selection Apparatus

The fourth embodiment of the present invention relates to an embryo selection apparatus for selecting a mammalian embryo prepared by in vitro culture from a fertilized egg. The apparatus of the present embodiment comprises an image shooting apparatus for obtaining an image of an embryo and an analysis section for analyzing the obtained image. The analysis section selects an embryo suitable for implantation based on the obtained image, provided that the embryo satisfies at least one of Index 1 to Index 3 below:
Index 1: a secretion product is not observed in the vicinity of an embryo at the blastocyst stage;
Index 2: partial compaction is not observed at the morula stage; and
Index 3: none of DC, AC, F, and UB is observed at a stage after first cleavage and before second cleavage.

The definitions of Index 1 to Index 3, methods of observation, and the like employed in the present embodiment are as described in the “[2] A method for selecting a mammalian embryo prepared by in vitro culture from a fertilized egg” section above, unless otherwise specified. It is preferable that the apparatus of the present embodiment select an embryo using Index 1. It is more preferable that the apparatus of the present embodiment select an embryo using Index 2 in addition to Index 1. It is more preferable that the apparatus of the present embodiment select an embryo using Index 3 in addition to Index 1. It is further preferable that the apparatus of the present embodiment select an embryo based on all of Index 1 to Index 3.

As the image shooting apparatus according to the present embodiment, a still image shooting apparatus, a moving image shooting apparatus, a time-lapse image shooting apparatus, or the like can be used. Use of a time-lapse image shooting apparatus that can obtain images of an embryo at each developmental stage regardless of individual differences is particularly preferable. It is preferable that a time-lapse image shooting apparatus be fixed inside a culture tank so as to obtain images of an embryo.

The apparatus according to the present embodiment comprises an analysis section that automatically evaluates as to whether or not an embryo of interest satisfies at least one of Index 1 to Index 3 and select an embryo suitable for implantation based on the images obtained. An analysis section may be equipped with a computer or the like. It is preferable that an analysis section perform two-step analysis: (I) analysis of an embryonic developmental stage; and (II) analysis of embryo conditions, based on the images. The analysis (I) evaluates the developmental stage of an embryo in the image, regardless of a normal/abnormal embryo. In accordance with the developmental stage evaluated, the analysis (II) of embryo conditions is performed. When an embryo is evaluated to be at a stage after first cleavage and before second cleavage by the analysis (I), the analysis (II) determines the occurrence of abnormal cleavage, such as DC, AC, F, or UB. When an embryo is evaluated to be at the morula stage by the analysis (I), the analysis (II) determines the occurrence of partial compaction. When an embryo is evaluated to be at the blastocyst stage by the analysis (I), the analysis (II) determines the presence or absence of a secretion product.

An analysis section may be equipped with artificial intelligence (AI). When an analysis section is equipped with AI, a machine-learned model using a known neural network may be preferably constructed with the use of the image databases of each developmental stage as teaching data for the analysis (I) and the image database of normal/abnormal embryo conditions at each developmental stage as teaching data for the analysis (II). With the use of such model, an analysis section can evaluate as to whether or not an embryo of interest in the image satisfies the indices. It is further preferable that the apparatus according to the present embodiment be equipped with an output section that further outputs the results of analysis.

### EXAMPLES

Hereafter, the present invention is described in greater detail with reference to the examples, although the present invention is not intended to limit the scope of the present invention to the scope of the examples.

### Test Example 1: Collection of Oocytes and In Vitro Maturation

Cattle cumulus-oocyte complex (COCs) collection and in vitro maturation were performed in accordance with the procedure described in the document of Imai et al. (Imai K, et al., J. Reprod., Dev. 52 (suppl.): 19-29, 2006). Ovaries collected from Japanese Black female cattle at a slaughterhouse were washed with physiological saline. COCs were aspirated from ovarian follicles (diameter: 2-6 mm) using 5- to 10-ml syringes with a 19-gauge needle, and they were then used for in vitro maturation. As a medium for in vitro culture, 25 mM Heptes buffer TCM199 (M199; Gibco BRL, Grand Island, N.Y., U.S.A.) supplemented with 5% calf serum and 0.02 IU/ml Antrin (Kyoritsu Seiyaku Corporation) was used. COCs were introduced into a tube and hermetically sealed with paraffin oil. The tube was introduced into a cell transporter (Fujihira Industry Co., Ltd.) set at 38.5° C. and transported to a culture chamber while culturing cells. After the tube reached the culture chamber, the tube was continuously subjected to culture for 22 hours after the initiation of in vitro maturation in a common culture vessel in a 5% CO₂ atmosphere and saturated humidity at 38.5° C.

### Test Example 2: In Vitro Fertilization

In vitro fertilization was performed in accordance with the procedure described in the above document of Imai et al. Specifically, Japanese Black bull sperm samples frozen in 0.5-ml straws were thawed in a water bath at 37° C. for 30 seconds, and then centrifuged in 3 ml of 90% Percoll solution at 2100×g for 10 minutes. Pellets were suspended again in a Brackett and Oliphant solution (BO solution) supplemented with 6 ml of a sperm washing solution (10 mM hypotaurine (Sigma), 2 U/ml heparin (Novo-Heparin Injection 1000; Aventis Pharma Ltd., Tokyo, Japan), and 10 mg/ml bovine serum albumin (BSA, crystallized and lyophilized; Sigma)). Centrifugation was performed to a final concentration of 3×10⁶ sperm cells/ml. Droplets (100 ml) of the suspension were formed on a 35-mm dish, the dish was coated with paraffin oil, and fertilization droplets were thus prepared. COCs were separated from the in vitro maturation medium, washed with a BO solution supplemented with 10 mg/ml BSA, and added to the fertilization droplets, so that each droplet contained 20 COCs. The resultants were cultured in a 5% CO₂ atmosphere and saturated humidity at 38.5° C. for 6 hours.

### Test Example 3: Time-Lapse Observation

A fertilized egg was cultured in the presence of 5% CO₂/5% O₂/90% N₂ and saturated humidity at 38.5° C. Development was observed using a culture vessel (WMI-165, ASTEC Co., Ltd.) comprising a time-lapse image shooting apparatus (see, for example, Patent Literature 3) mounted thereon. Images of a fertilized egg were obtained at intervals of 15 minutes for 7 to 8 days. On the basis of the images obtained, evaluation was performed in terms of the indices below:
Index 1: the presence or absence of a secretion product from a blastocyst;
Index 2: occurrence of partial compaction at the morula stage; and
Index 3: the form of first cleavage (occurrence of direct cleavage (DC), abnormal cytokinesis (AC), fragmentation (F), or unequal sizes among sister blastomeres (UB)).

### Test Example 4: Implantation of Fertilized Egg

Holstein or Japanese Black cattle was used as a recipient. A fertilized egg 7 to 8 days after culture was transplanted to the luteal uterine horn of a recipient in estrus or a recipient whose sexual cycle had been synchronized.

### Test Example 5: Diagnosis of Conception

On 30 days and 60 days after embryo implantation, conception diagnosis was performed by ultrasonography. Conception was confirmed by observing intrauterine fetuses and confirming fetal heartbeats.

### Test Example 6: Analysis of Correlation Between an Index and a Conception Rate

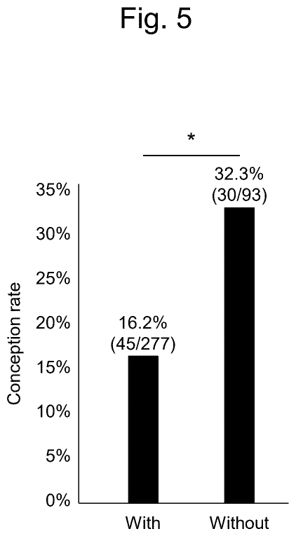

Whether or not Index 1 to Index 3 are significantly correlated with conception rates was analyzed. FIG. 5 shows a chart showing conception rates of the tested 370 samples classified into: a group of embryos not satisfying Index 1 (a group of embryos “with” a secretion product); and a group of embryos satisfying Index 1 (a group of embryos “without” a secretion product). In FIG. 5, the symbol “*” indicates that the p-value exceeds 0.05 in a χ² test (P<0.05). Comparison of two groups demonstrates that a group satisfying Index 1 exhibits significantly high conception rates.

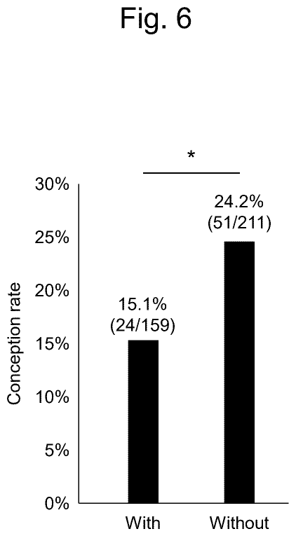

FIG. 6 shows a chart showing conception rates of the tested 370 samples classified into: a group of embryos not satisfying Index 2 (a group of embryos “with” PC); and a group of embryos satisfying Index 2 (a group of embryos “without” PC). In FIG. 6, the symbol indicates that the p-value exceeds 0.05 in a χ² test (P<0.05). Comparison of two groups demonstrates that a group satisfying Index 2 exhibits significantly high conception rates.

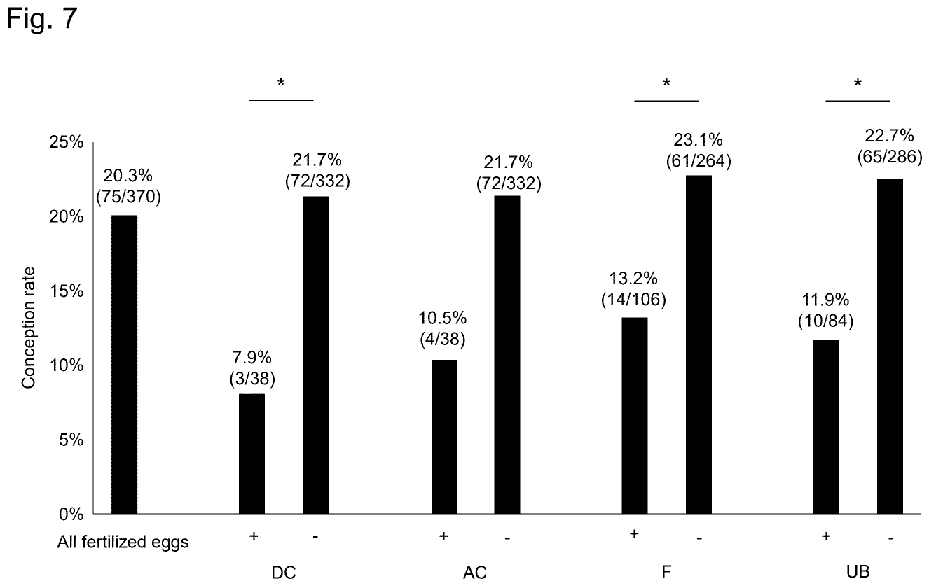

FIG. 7 shows a chart showing conception rates of the tested 370 samples that are classified into: a group of embryos that undergo DC, AC, F, and UB after first cleavage (a group of embryos “+”); and a group of embryos that do not undergo DC, AC, F, and UB (a group of embryos “−”). In FIG. 7, the symbol “*” indicates that the p-value exceeds 0.05 in a χ² test (P<0.05). The group of embryos “−” was found to exhibit higher conception rates than those of the group of embryos “+” with the use of any of DC, AC, F, and UB as the index.

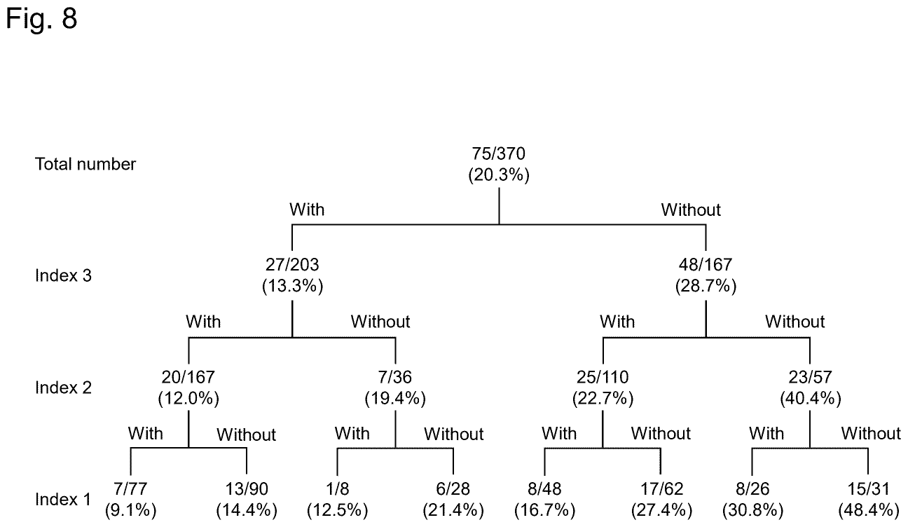

Table 1 shows the number of embryos satisfying the indices and the conception rates thereof when embryos are evaluated with the use of one or more of Index 1 to Index 3. FIG. 8 shows conception rates of fertilized eggs (embryos) evaluated in the order from Index 3, Index 2, to Index 1. As shown in Table 1 and FIG. 8, the highest conception rate (48%) was achieved by selecting embryos with the use of Index 1 in combination with Index 2 and Index 3. This conception rate (48%) was more than double the conception rate (20%) achieved upon implantation of code 1 embryos in accordance with the IETS standards (conventional standards).

TABLE 1

| | The number of | The number | The number |
|---|---|---|---|
| | implanted fertilized | of fertilized | of unfertilized |
| Index | eggs satisfying the index | eggs (%) | eggs (%) |
| Index 1 | 211 | 51 (24) | 160 (76) |
| Index 2 | 90 | 30 (33) | 60 (67) |
| Index 3 | 167 | 48 (29) | 119 (71) |
| Index 1 and Index 2 | 56 | 21 (38) | 35 (62) |
| Index 1 and Index 3 | 93 | 32 (34) | 61 (66) |
| Index 2 and Index 3 | 57 | 23 (40) | 34 (60) |
| Index 1, Index 2, and Index 3 | 31 | 15 (48) | 16 (52) |
| IETS (Code1) | 187 | 37 (20) | 150 (80) |
| Total number | 370 | 75 (20) | 295 (80) |

All publications, patents, and patent applications cited herein are incorporated herein by reference in their entirety.

1. A method for selecting a mammalian embryo prepared by in vitro culture from a fertilized egg comprising selecting an embryo, provided that the embryo's appearance satisfies at least one of the Index 1 to Index 3 below:
Index 1: a secretion product is not observed in the vicinity of an embryo at the blastocyst stage;
Index 2: partial compaction is not observed at the morula stage; and
Index 3: none of direct cleavage (DC), abnormal cytokinesis (AC), fragmentation (F), and unequal blastomeres (UB) is observed at a stage after first cleavage and before second cleavage.

2. The method according to claim 1, wherein the selecting an embryo satisfies Index 1.

3. The method according to claim 2, wherein the selecting an embryo further satisfies Index 2.

4. The method according to claim 2, wherein the selecting an embryo further satisfies Index 3.

5. The method according to claim 1, wherein the selecting an embryo satisfies all of Index 1 to Index 3.

6. The method according to claim 1, wherein the mammal is cattle.

7. A method for producing an embryo from a mammalian fertilized egg comprising:
in vitro culture of a fertilized egg; and
at least one of Step a to Step c below:
Step a: selecting an embryo that does not have a secretion product in the vicinity thereof at the blastocyst stage;
Step b: selecting an embryo that does not undergo partial compaction at the morula stage; and
Step c: selecting an embryo that does not undergo any of direct cleavage (DC), abnormal cytokinesis (AC), fragmentation (F), or unequal blastomeres (UB) at a stage after first cleavage and before second cleavage.

8. The method according to claim 7, which comprises Step a.

9. The method according to claim 8, which further comprises Step b.

10. The method according to claim 8, which further comprises Step c.

11. The method according to claim 7, which comprises all of Step a to Step c.

12. The method according to claim 7, wherein the mammal is cattle.

13. A method for producing an individual mammal comprising a step of implanting an embryo prepared by the method according to claim 7 into a female for conception.

14. An embryo selection apparatus for selecting a mammalian embryo prepared by in vitro culture from a fertilized egg comprising:
an image shooting apparatus for obtaining an image of an embryo; and
an analysis section for analyzing the obtained image,
wherein the analysis section selects an embryo suitable for implantation based on the obtained image, provided that the embryo satisfies at least one of Index 1 to Index 3 below:
Index 1: a secretion product is not observed in the vicinity of an embryo at the blastocyst stage;
Index 2: partial compaction is not observed at the morula stage; and
Index 3: none of direct cleavage (DC), abnormal cytokinesis (AC), fragmentation (F), and unequal blastomeres (UB) is observed at a stage after first cleavage and before second cleavage.
